(19) United States
(12) Patent Application Publication
(10) Pub. No.: US 2013/0261142 A1
(43) Pub. Date: Oct. 3, 2013

(54) COMPOUNDS USED FOR TREATING CANCER AND THE USE THEREOF

(71) Applicant: Hung-Cheng Lai, Taipei City (TW)

(72) Inventor: Hung-Cheng Lai, Taipei City (TW)

(21) Appl. No.: 13/992,633

(22) PCT Filed: Dec. 15, 2010
(86) PCT No.: PCT/CN2010/079835

§ 371 Date: Jun. 7, 2013

(51) Int. Cl. A61K 31/167

(52) U.S. Cl. CPC A61K 31/167
USPC 514/280; 514/622; 514/456

(57) ABSTRACT

The present invention discloses compounds for the treatment of cancer and its application. These compounds comprises one of the following compound: Ammonium pyrrolidinedithiocarbamate [symbol] Bay 11-7085 [symbol] BIO [symbol] Brefeldin A [symbol] (+)-Butaclamol [symbol] Calcimycin [symbol] Calmidazoliur chloride [symbol] Chelerythrine chloride [symbol] CK2 Inhibitor 2 [symbol] CGP-74514A hydrochloride [symbol] CGS-12066A meleate [symbol] Dequalinium dichloride [symbol] Dihydroouabain [symbol] Diphenyleneiodonium chloride [symbol] Emetine dihydrochloride hydrate [symbol] GR 127935 hydrochloride [symbol] Nifedipine [symbol] 6-Nitroso-1,2-benzopyrone [symbol] Palmitoyl-DL-Carnitine chloride [symbol] Parthenolide [symbol] PD 169316 [symbol] 1,10-Phenanthroline monohydrate [symbol] 4-Phenyl-3-furoxancarbonitrile [symbol] Prazosin hydrochloride [symbol] Protoporphyrin IX disodium [symbol] Quinacrine dihydrochloride [symbol] Quabain [symbol] Retinoic acid p-hydroxyanilide [symbol] Rottlerin [symbol] Sanguinarine chloride [symbol] Tetraethylthium disulfide and SU 9516. The invention also provides new uses of these compounds, compounds such as for the preparation of the treatment of cancer, inhibit cancer cell, cancer stem cell growth and provides a new pharmaceutical composition for treating cancers

### BACKGROUND OF THE INVENTION

1. Field of the Invention

The present invention relates to a method for treating cancer cells and cancer stem cells by screening compounds capable of inhibiting spheroid differentiation as well as growth of cancer cells from the LOPAC database.

2. Description of the Prior Art

Due to the explosive stem cell research in the past decade, a hypothesis of cancer stem cells has gradually emerged, stating mistakes generated during the process of self-renewal in stem cells or progenitor cells in tissues give rise to abnormal cells, and the resulted cells exhibit the properties of stem cells, including self-renewal and differentiation, contributing to the formation of tumors. According to clinical evidence provided by the theory of disease progression, the atomic bombs dropped by the United States in Hiroshima and Nagasaki in Japan in August 1945 caused significant casualties, including countless girls in puberty stage, and the follow-up study conducted 20 to 30 years later indicated that nearly all the females were diagnosed with breast cancer. Since the mutations found in those cancer cells are induced by radiation, it is speculated that radiation exposure may contribute to the mutations of the stem cells in the breast tissues of these females during their puberty stage while numerous stem cells existed, which consequently caused breast cancer later on. Further studies also demonstrated that stem cells play a crucial role in the process of tumorigenesis because normal stem cells share a number of similarities with the cancer cells such as both are capable of self-renewal and differentiation, express activated telomerase, can activate anti-apoptotic pathway, can increase membrane transportation as well as possess the potential to spread and to metastasize to another place in the body. Researchers speculated that loss of regulation during the self-renewal process of stem cells may be one of the reasons that induces the process of tumorigenesis in early stage. Thus far, various pathways involved in regulation of the self-renewal process of stem cells have been discovered, for instance, Wnt, Notch and Hedgehog pathways, etc. From previous studies, these pathways play key roles in the process of tumorigenesis as well, for example, defect in the Wnt pathway was observed in early stage of colon cancer, and deficiency of the Hedgehog pathway was found in the basal cell carcinoma of skin. Moreover, the Notch gene may also play a role in the process of colon cancer tumorigenesis due to induction of tumor growth. Cellular signaling transduction pathway ensures the balance of the abovementioned mechanisms, and Notch as well as Wnt are genes that regulate the cellular signaling transduction pathway which assures normal development of intestine and other tissues. These two genes start to function as the stem cells in intestine begin to proliferate and differentiate. The APC gene is located at the upstream of the Wnt signaling pathway, and its mutations are commonly considered as the causes of colon cancer. Mutations in one of the two alleles of the APC gene result in familial adenomatous polyposis (FAP). The feature of the disease is the presence of hundreds or even thousands of adenomatous polyps in patient's colon, and 1% of colon cancer cases were developed from these polyps. In addition, defect of the Hedgehog pathway was also found in pancreatic cancer, stomach cancer, prostate cancer and breast cancer, while deficiency of the Notch pathway was observed in acute T cell leukemia, cervical cancer as well as breast cancer. Based on the studies published in the past few years, several common target proteins were identified in cancer stem cells collected from various cancers, including CD44+, α6 integrin+, β1 integrin+, CD133+ (Prominin) and ALDH+.

Ovarian cancer is one of the gynecological cancers with the highest mortality rate. Ovary is located in the pelvic cavity, and unless the tumor is large enough to be detected by abdominal palpation, ovarian cancer is usually not easily detected. The most common symptoms of ovarian cancer are abdominal discomfort, nausea, anorexia, similar to the symptoms of gastrointestinal diseases. Ovarian cancer is a common gynecological cancer in the U.S., and its incidence and mortality rate have remained approximately constant in North America and Western Europe (Bray et al., 2005; Jemal et al., 2008) while increasing trends were observed in previously low-risk areas in southern and eastern Europe, and Asia (Bray et al., 2005; Tamakoshi et al., 2001; Yen et al., 2003; Yeole, 2008). Due to the fact that recurrence of ovarian cancer is extremely high as well as easy metastasis of the ovarian cancer cells, most patients were diagnosed at advanced stage upon recurrence, thereby more medical resource is required to treat patients with ovarian cancer while the mortality rate remains high (Jemal, Siegel et al. 2008). The treatments for ovarian cancer mainly include traditional methods such as surgical removal of the cancerous tissue; radiation therapy to shrink solid tumor; and chemotherapy that kills cancer cells rapidly, but all with limited success (Cannistra 2004). It is also surprised to find that the mortality rate of ovarian cancer remained high over the past decade (Oriel Hartenbach et al 1999; Jemal, Siegel et al 2008). Due to the lack of data analysis of the population as well as the high mortality rate, delays in treatment, along with no effective early warning mechanisms, the incidence, mortality and popularity of ovarian cancer have shown an increasing trend.

According to the data obtained from previous tumor analysis studies, the incidence, growth, and differentiation of ovarian cancer vary significantly from sample to sample, therefore triggered the researchers to consider whether the function and mechanism of tumor stem cells attribute to the differences observed in cancer cells (Reya, Morrison et al. 2001). Normal stem cells are capable of self-renewal indefinitely and can further differentiate into numerous cells or tissues. The difference between embryonic stem cells and adult stem cells is the plasticity, and relatively speaking, a significant portion of the somatic cells are with limited capability to self-renew because the telomere is shortened in most somatic cells. Previous studies have suggested that transfer of a single undifferentiated leukemia cell to mice caused leukemia in the transgenic mice. Cancer stem cells are obtained from cancerous tissues or cell groups. At present, cancer stem cells (CSCs) are considered very similar to common adult stem cells in that they share the same molecular markers and cell morphology; additionally, hematopoietic stein cells were found to express CD34, CD90 and CD133 on their surface after long-term study. Confirmation of hematopoietic stem cells using CD34 and CD133 surface antigens as well as ATP-binding cassette sub-family G member 2 (ABCG2) suggested that ABCG2 is highly correlated with side population cancer cells, and ABCG2 is highly expressed on the cell surface of the stem cells obtained from various sources, indicating ABCG2 is very likely involved in the process of stem cell differentiation and the location of mesenchymal stem cells in human. The adenine will exclude the Hoechst dye 33342 from nucleus, thereby flow cytometry can be used to identify stem cells. Hoechst 33342 staining is an quantitative method as well as a fluorescent staining method used for measurement of DNA concentration, and the labeled DNA as well as the position of the DNA-containing nucleus and mitochondria can be observed under a fluorescence microscope by Hoechst staining. After observation and comparison the data from DNA staining, cells with the stem cell potential can be further isolated from normal cells. On the other hand, cells will not cause tumorigenesis in other cells; however, tumorigenic cells will induce tumor formation of the cells nearby. Cancer stem cells are different from cancer cells and common human stem cells; nonetheless, those cells remain capable of differentiation, proliferation and self-renewal as well. The discovery and concepts of cancer stem cells were proposed in 1960, stating that in vitro cultured cancer stem cells are not only capable of continuous differentiation and proliferation, but can form tumors in vivo as well. Studies afterwards have successfully identified the cancer stem cells with stem cell characteristics from acute myeloid leukemia. Moreover, in 2003, scientists further isolated cancer stem cells with stem cell properties from breast tumor. Since then, more side population cancer cells with cancer stem cell features have been found from various cancers, including brain tumor, melanoma, lung cancer, prostate cancer, pancreatic cancer, head and neck cancer, colon cancer, liver cancer and advanced ovarian cancer. Cancer stem cells are one of the main factors that result in unsuccessful chemotherapy and radiation therapy. Therefore, understanding the metastasis process of cancer stem cells and relevant mechanisms may affect the judgment and treatment methods for cancer diagnosis and treatment.

Due to the number of cancer stem cells in a tissue or a group of cells is very scarce and those cells lack of the morphology found in common cancers, identification of the functions of cancer stem cells is rather difficult. However, Hoechst staining can be used as a stem cell probe to identify the transportation pores on cell membrane, for example, ABC transporters (e.g. ATP-binding cassette); Multidrug resistance protein (MDR1); Breast Cancer Resistance Protein 1 (BCRP1); and ATP binding cassette, sub-family G, and member 2 (ABCG2). Studies have proved that these factors are expressed in cancer stem cells. Moreover, expression of Bcrp1/ABCG further highlights the stem cell properties. Hence, using Hoechst staining for identification of the activity of cancer stem cells from side population cancer cells is a significant representative of present invention.

In addition to the abovementioned markers, the selective marker-free approach for labeling genes may also be used for studying the differentiation of cell spheres and help to understand the differentiation process and pathways of the undifferentiated and pluripotent sphere stem cells. In general, 4-20% of these sphere stem cells are stem cells that are at various undifferentiated and quiescence stages; yet, these cells are pluripotent. Similar studies also demonstrated that nonadherent spheroids with stem cell properties were isolated from breast tissue and human cancer tissues. These pluripotent cancer stem cells were stained positive when examined by common cancer stem cell markers such as ABCG2, CD117, and CD133.

In the field of ovarian cancer research, studies have shown that the subclones of spheroids can be produced from ascites collected from ovarian cancer patients, and the gene characteristics as well as protein expression, including OCT4, NANOG, and CD133, can also be used for detection of cancer stem cell expression. OCT4 is one of the important transcription factors expressed during the early stage of mammalian embryo development and is only expressed in pluripotent cells; therefore, OCT4 is involved in the process of cell differentiation. Likewise, cancer stem cells with CD133 expression are also found in certain cancer cells, and along with OCT4 and NANOG these markers can be used for diagnosis of proliferation or metastasis of the cancer and determine the prognosis of cancer patients. Thus far, the cancer stem cells were selected by staining of mammalian side population cancer cells, and the ovarian cancer progenitor cells were also successfully isolated from the spheroids derived from the ovarian cancer sphere cells using CD44/CD117 markers. The sort of techniques are more advantageous for development of effective new drugs for treatment. Combination of the dye-exclusion technique and spheroid formation assays can improve the isolation results of ovarian cancer progenitor cells. Most importantly, the most effective drugs against the progenitor cells of ovarian cancer or other cancers can be selected by High Throughput Drug Screening technology as well.

By far, the activity of cancer stem cells is considered the main reason of cancer recurrence and metastasis. Traditional therapies for treating cancer involve surgical removal of cancerous tissues, chemotherapy, and radiotherapy so as to reduce the number of cancer cells. However, uninhibited activity of cancer stem cells can remain contribute to cancer recurrence and metastastasis. Hence, the common traditional methods for treating cancers have a number of disadvantages and usually with high metastasis and recurrence, thereby these methods evidently are not the best solution for cancer treatment. A more direct treatment approach for early detection and early therapy is needed.

After years of research and development, the inventor(s) of the present invention successfully developed the compound for cancer treatment and disclosed its applications thereof.

### SUMMARY OF THE INVENTION

The present invention provides a previously discovered compound and used the compound as an anti-cancer drug to interfere with the differentiation and proliferation of cancer stem cells. The inhibition effect(s) of the compound on cancer stem cells and the mechanism(s) of the inhibition effect(s) were examined by administration of Ammonium pyrrolidinedithiocarbamate; Bay 11-7085; (2′Z,3′E)-6-Bromoindirubin-3′-oxime (BIO); Brefeldin A; (+)-Butaclamol; Calcimycin; Calmidazolium chloride; Chelerythrine chloride; CK2 Inhibitor 2; CGP-74514A hydrochloride; CGS-12066A meleate; Dequalinium dichloride; Dihydroouabain; Diphenyleneiodonium chloride; Dihydroouabain; Diphenyleneiodonium chloride; Emetine dihydrochloride hydrate; GR 127935 hydrochloride; beta-Lapachone; Niclosamide; Nifedipine; 6-Nitroso-1,2-benzopyrone; Palmitoyl-DL-Carnitine chloride; Parthenolide; 1,10-Phenanthroline monohydrate; 4-Phenyl-3-furoxancarbonitrile; Prazosin hydrochloride; Protoporphyrin IX disodium; Quinacrine dihydrochloride; Quabain; Retinoic acid p-hydroxyanilide; Rottlerin; Sanguinarine chloride; Tetraethylthium disulfide and SU 9516. The compound can be used as a single treatment or be combined with other anti-cancer therapies.

In particular, the abovementioned compound can trigger and result in apoptosis of cancer stem cells when added to cancer stem cells and examined by Hoechst 33342 staining, flow cytometry, area analysis, proliferation dynamics, image-based screen assay, MTS assay and preclinical animal study.

In one aspect, the present invention provides a method using the aforementioned compound or its pharmaceutically acceptable salts, solvates or pharmaceutically functional derivatives to inhibit cancer cells and cancer stem cells, wherein the cancer is one of the following cancers including cervical cancer; breast cancer; colon cancer; pancreatic cancer; stomach cancer; prostate cancer; acute T cell leukemia; leukemia; liver cancer; endometrial cancer; lung cancer; colorectal cancer; melanoma or malignant sarcoma.

In another aspect, the pharmaceutical composition used for inhibition the growth of cancer cells of the present invention can be added to pharmaceutically acceptable diluents, fillers, binders, disintegrants, and lubricants in various dosage forms for treating cancer in clinical medicine.

These features and advantages of the present invention will be fully understood and appreciated from the following detailed description of the accompanying Drawings.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the preferred embodiments shown.

In the drawings:

FIG. 1A shows the Hoechst 33342 staining results of four different cancer cell lines.

FIG. 1B shows the inhibition effect of ABCG2 on cancer stem cells, wherein the result indicates that the ABCG2 inhibitor significantly reduces the number of cancer stem cells.

FIG. 1C shows the variations in sizes of various cancer stem cell lines.

FIG. 1D demonstrates the formation of spheroid, wherein the spheroid was from by culturing cancer stem cells on a culture dish.

FIG. 2A shows the marker expression of cancer stem cells including NANOG, OCT4, and NESTIN, using flow cytometry.

FIG. 2B shows the expression of three cell surface markers (CD34, CD44, CD133) in ovarian cancer cells (CP70) as well as in ovarian cancer stem cells (CP70SPS).

FIG. 3A shows the Hoechst 33342 staining result of MCF-7 sps cells treated with ABCG2 inhibitor, wherein the result indicated that the cell number is significantly reduced after treated with ABCG2 inhibitor.

FIG. 3B shows CD24 and CD44 expression on the cell surface of breast cancer stem cells, MCF-7 sps.

FIG. 3C shows NANOG, OCT4, NESTIN and ABCG2 expression in cancer stem cells by protein marker analysis using flow cytometry.

FIG. 3D shows formation of the spheroid, wherein the spheroid was from by culturing cancer stem cells on a culture dish.

FIG. 4A shows tumors induced by injection of rats with MCF-7 SPS.

FIG. 4B shows the cytotoxicity of anti-cancer drug, Paclitaxol, on breast cancer stem cells (MCF-7 SPS), human mammalian epithelial carcinoma cells (MDA-MB 231) and human mammalian epithelial carcinoma stem cells (MDA-MB 231 sps).

FIG. 5A demonstrates the inhibition effects of the signaling transduction inhibitors, including 2.1 mg/cc DKK, 30 μM GSI and 30 μM cyclopamide, wherein the inhibition levels of Wnt, NOTCH and shh were examined 3 days post treatment.

FIG. 5B shows the aggregates formed by cancer stem cells, which subsequently induce the formation of tumor spheroid after been treated with the NOTCH inhibitor, 30 μM GSI.

FIG. 6A shows the inhibition effect of Niclosamide on breast cancer stem cells, MCF-7 sps, wherein the inhibition was significant.

FIG. 6B demonstrates the inhibition effects of Niclosamide on breast cancer stem cells (MCF-7 sps) and human mammalian epithelial carcinoma cells (MDA-MB 231).

FIG. 7A shows the inhibition effects of Niclosamide on breast cancer-correlated binding protein (Cyclin D1), cervical cancer-correlated binding protein (Hes-1) and pancreatic cancer-correlated binding protein (PTCH).

FIG. 7B shows the cell survival rates of MCF-7, MCF-7 sps and MDA-MB 231 sps following Niclosamide treatment.

FIG. 8A shows the change of the tumors following Niclosamide treatment in rats with induced breast cancer, wherein the results indicated that the change was significant in the treated rats.

FIG. 8B represents the weight variations in rats with induced breast cancer following Niclosamide treatment, wherein the results indicated that the weights of the treated rats were significantly different.

FIG. 9 shows the ALDH-1 expression in various cancer cell lines.

FIG. 10A shows the cell vitalities of breast cancer stem cells and breast cancer cells.

FIG. 10B shows the numbers of survived CP70 and CP70SPS cells following Cisplatin treatment.

FIG. 10C shows the survival rates of CP70 cells at different differentiation stages following anti-cancer drug, Cisplatin (CDDP), treatment.

FIG. 11A shows the tumors induced by injection of rats with CP70 sps cells.

FIG. 11B shows the tumors induced by injection of rats with CP70 cells.

FIG. 12 shows the inhibition effects of Niclosamide, Rottlerin, and (+)-Butaclamol hydrochloride on CP70 and CP70 SPS cells as well as the correlated cell survival rates.

FIG. 13A shows the inhibition effect of Niclosamide on rats with induced ovarian cancer, wherein the results indicated that the tumors were significantly different in the treated rats.

FIG. 13B shows the numbers of ovarian cancer cells in the tumors obtained from Niclosamide-treated rats, wherein the results indicated that the cancer cells were inhibited.

FIG. 14 shows the numbers of ovarian cancer stem cells found in CP70 SPS cell culture in vitro following Niclosamide treatment, wherein the results indicated that CP70 SPS cells were inhibited.

FIG. 15 shows the numbers of ovarian cancer stem cells found in CP70 SPS cell culture in vitro following (+)-Butaclamol hydrochloride treatment, wherein the results indicated that CP70 SPS cells were inhibited.

### DETAILED DESCRIPTION OF THE PREFERRED EMBODIMENT

### Example 1

### 1. Experimental Materials

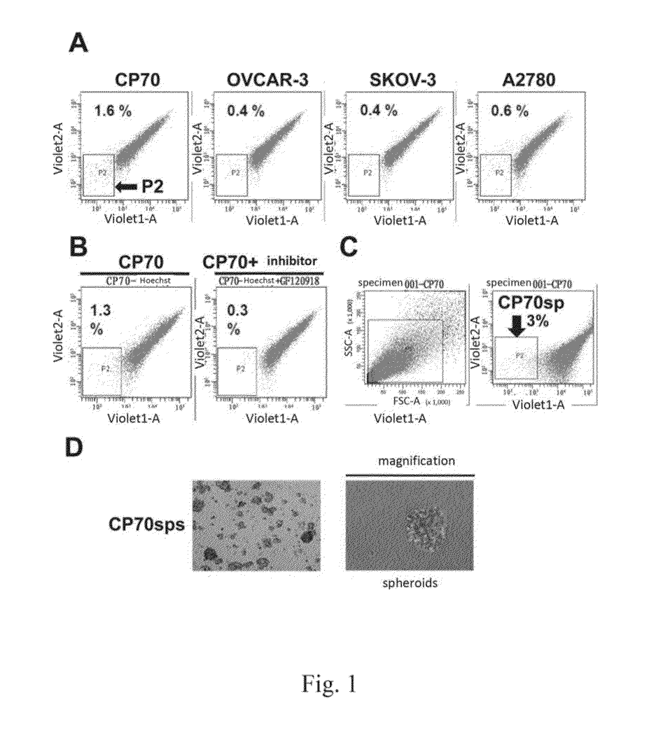

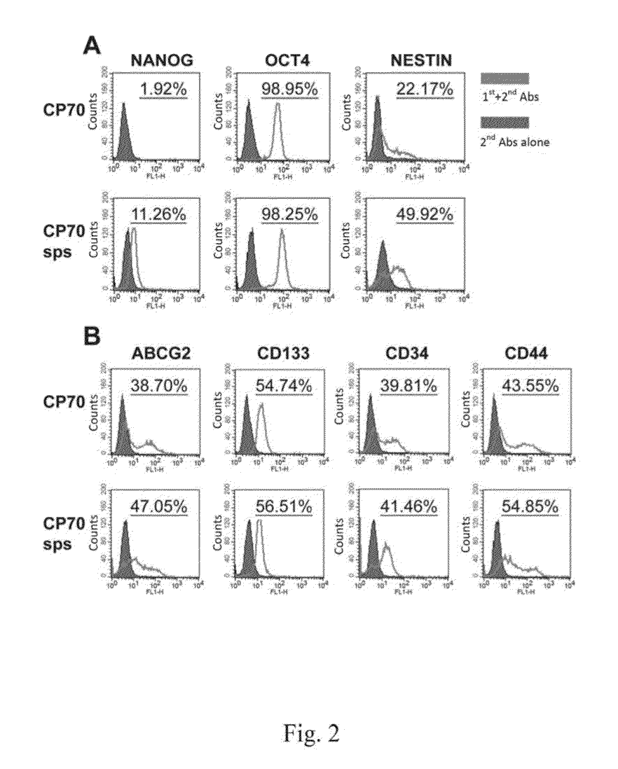

The ovarian cancer cells (CP70) and ovarian stem cancer cells (CP70 sps) are used, and CP70 cells were stained by Hoechst dye 33342 for screening of the side population cancer cells. The selected cells were then cultured in cancer stem cell culture media to induce differentiation and resulted cells are ovarian cancer stem cell, CP70 sps. To further verify the obtained ovarian cancer stem cells, various cell markers were stained, including NANOG, OCT4, NESTIN (an embryonic stem cell marker), ABCG2, CD34, CD44, CD133 and ALDH1. The results indicated that all markers showed increased expression (FIGS. 1A, 1B, 1C, 2A, and 2B). Additionally, the ovarian cancer stem cells (CP70 sps) formed spheroid (FIG. 1D).

Confirmation of the Stem Cell Differentiation Properties of the Ovarian Cancer Stem Cells CP70 sps

CP70 sps P0 cells were injected intraperitoneally (i.p.) into NOD/SCID mice. After 70 days, tumor nodules were harvested as CP70spsP1 cells. For comparison of tumorigenic capacity, NOD/SCID mice were inoculated i.p. with various numbers of CP70, CP70sps P0, and CP70sps P1 cells, and were monitored throughout the experimental period. The mice with abdominal swelling were sacrificed and ascites as well as the size, weight, shape of the tumor formed were recorded.

### 2. Screening of the Bioactive Compound

CP70 sps cells were seeded in a 96-well culture plate (1×10³ cells/well) and cultured for 24 hr, and then treated with 30 μM of various compounds selected from the LOPAC database. Following 3-day treatment, the vitality of the treated cells was measured by CellTiter-Glo® Luminescent Cell Vitality Assay kit (Promega) according to the manufacturer's instructions.

### 3. Detection of the Bioactive Compounds that are Capable of Inhibition of Cancer Stem Cell Differentiation

A total of 61 screened compounds from the LOPAC database were found to inhibit the growth of CP70 sps cells (Table 1). To further examine the inhibition effects of these compounds, 5000 CP70 sps cells were seeded per well in a 96-well culture plate for 24 hours and then treated separately with a lower concentration (3 μM) of the 61 selected compounds. The vitality of the treated cells was measured by MTS assay using MTS-based CellTiter 96 aqueous non-radioactive cell proliferation assay kit (Promega) according to the manufacturer's instructions. From the results, Niclosamide, Rottlerin and (+)-butaclamol hydrochloride all showed significant inhibition effects.

TABLE 1
Various bioactive compounds in the LOPAC database

| Class | Total agent ID | Selective agent |
|---|---|---|
| Cytotoxic agent | 91 | 10 |
| Biochemistry | 43 | 4 |
| Adenosine | 53 | 1 |
| Adrenoceptor | 102 | 2 |
| Hormone | 31 | 1 |
| Cholinergic | 77 | 1 |
| Somatostain | 2 | 1 |
| Neurotransmitter | 423 | 14 |
| Intracellular Calcium | 7 | 3 |
| Ion pump and ion channel | 72 | 7 |
| Multi-Drug Resistance | 12 | 2 |
| Nitric Oxide | 36 | 3 |
| Phosphorylation | 92 | 8 |
| Tachykinin | 5 | 1 |
| | 1258 | 61 |

### 4. The Inhibition Model of Niclosamide in CP70 sps Cells

CP70 sps cells were seeded in a 96-well plate at 5×10³ per well and treated separately with different concentrations of Niclosamide, ranging from 2.25 μM to 18 μM in duplicates for 3 days. The vitality of the CP70 sps cells was measured by MTS assay. For preclinical test, two groups with five mice each were i.p. injected with 1×10⁴ of CP70 sps cells alone or followed by Niclosamide treatment (10 mg/kg/daily) from day 1 to day 47, respectively. The body weights of the two groups of mice were monitored every 3 days during day 36 to day 48. After 48 days, the mice were sacrificed to examine the tumor nodules formation in the abdominal cavity.

### 5. The Inhibition Effect of Niclosamide on Patients' OVCA Specimen-Derived Cells (pdOVCICs)

A total of 5000 pdOVCICs were seeded onto each well in 96-well plates and each plate was treated with different concentrations of Niclosamide in duplicates for 3 days. The vitality of pdOVCICs cells was measured by MTS assay.

### Example 2

### Differentiation of MCF-7 Cells into MCF-7sps Cells and Inhibition Effect of Niclosamide

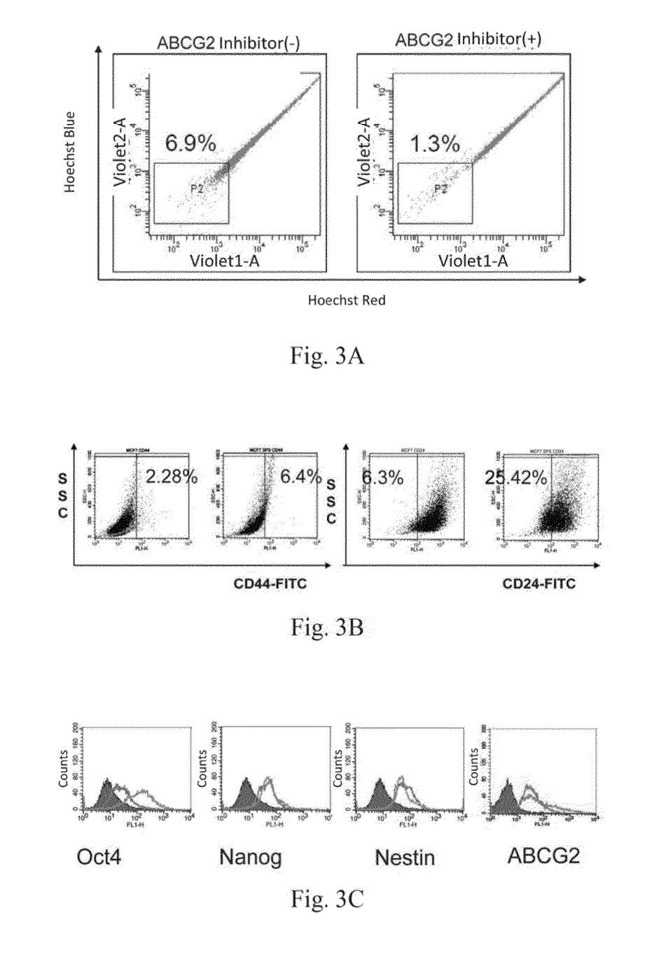

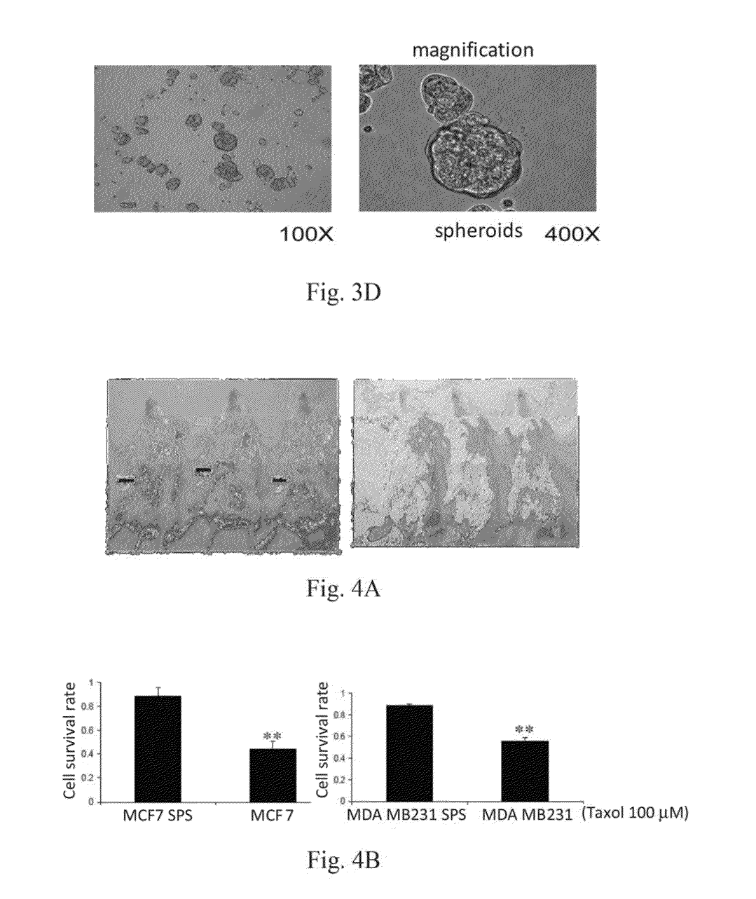

Hoechst dye 33342 was used for screening of side population cancer cells from MCF-7 cells followed by formation of MCF-7 sps cells in cancer stem cell culture medium from the selected cells. To further confirm the characteristics of the cultured MCF-7 sps cells, the cells were examined by various cell surface markers, including embryonic stem cell markers, NANOG, OCT4 and NESTIN, as well as cancer stem cell markers such as ABCG2, CD24 and CD44, which all showed increased expression (as shown in FIGS. 3A, 3B and 3C) and the Cp70 sps cells formed spheroid as indicated in FIG. 3D.

MCF-7 cells and MCF-7 sps cells were then compared by subcutaneous injection of these two cell lines into NOD/SCID mice, and the differences observed during the experimental period was recorded. The NOD/SCID mice with tumor formation were sacrificed and the size, weight and shape of the tumor formed were recorded as well. From FIG. 4A, injection of MCF7 sps cells into NOD/SCID mice gave rise to tumors in these mice; but no tumor was found in the mice injected with MCF-7 cells.

Next, we compared the toxicity effects of the anti-cancer drug, Paclitaxol, on MCF-7 cells, MCF-7 SPS cells, MDA-MB 231 cells and MDA-MB 231 SPS. The results indicated that MCF-7 cells were notably inhibited by Paclitaxol whereas the drug had no effect on MCF-7 sps cells (FIG. 4B).

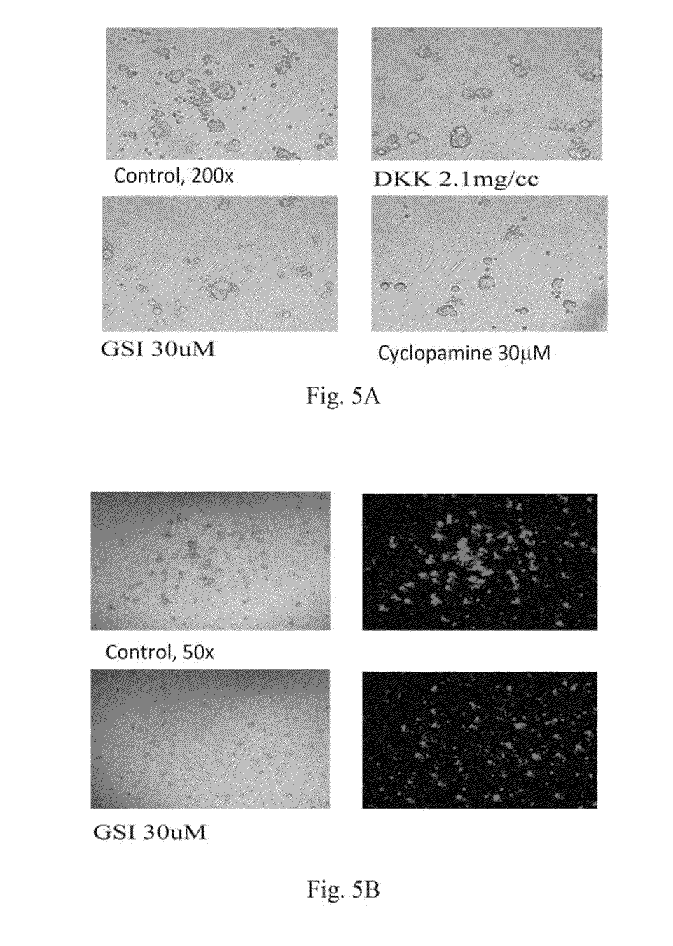

FIG. 5A demonstrates that the expression of Wnt, NOTCH and shh were inhibited by treating the MCF-7 SPS cells with various signaling inhibitors: 2.1 mg/cc DKK, 30 μM GSI or 30 μM cyclopamide for 3 days. Moreover, MCF-7 SPS cells were affected by the expression of Wnt, NOTCH and shh which caused morphological changes. On the other hand, treatment of the NOTCH inhibitor, 30 μM GSI, led to aggregation of the cancer stem cells and consequently resulted in formation of tumor spheroid. Based on the aforementioned experiments, MCF-7 SPS cells contain cancer stem cells that can undergo further differentiation (FIG. 5B).

A group of 1258 compounds that can potentially inhibit differentiation and growth of MCF-7 SPS cells were selected from the LOPAC database and tested for their inhibition effects on differentiation. After screening, 30 compounds including Ammonium pyrrolidinedithiocarbamate, (E)3-((4-t-Butylphenyl)sulfonyl)-2-propenenitrile)(Bay 11-7085), (2′Z,3′E)-6-Bromoindirubin-3′-oxime)(BIO), γ-4-Dihydroxy-2-(6-hydroxy-1-heptenyl)-4-cyclopentanecrotonic acid λ-lactone) (Brefeldin A), (+)-Butaclamol, Calcimycin, 1-[bis(4-Chlorophenyl)methyl]-3-[2-(2,4-dichlorophenyl)-2-(2,4-dichlorobenzyloxy)ethyl]-1H-imidazolium chloride) (Calmidazolium chloride), Chelerythrine chloride, 4,5,6,7-tetrabromobenzimidazole (CK2 Inhibitor 2), N2-(cis-2-Aminocyclohexyl)-N6-(3-chlorophenyl)-9-ethyl-9H-purine-2,6-diamine hydrochloride)(CGP-74514A hydrochloride, 7-Trifluoromethyl-4-(4-methyl-1-piperazinyl)pyrrolo-[1,2-a]quinoxaline maleate salt)(CGS-12066A meleate), Dequalinium dichloride, Dihydroouabain, Diphenyleneiodonium chloride, Emetine dihydrochloride hydrate, N-[4-Methoxy-3-(4-methyl-1-piperazinyl)phenyl]-2′-methyl-4′-(5-methyl-1,2,4-oxadiazol-3-yl)-1,1′-biphenyl-4-carboxamide hydro chloride hydrate)(GR 127935 hydrochloride), 3,4-Dihydro-2,2-Dimethyl-2H-Naphtho[1,2-B] Pyran-5,6-Dione)(beta-Lapachone), Niclosamide, Nifedipine, 6-Nitroso-1,2-benzopyrone, Palmitoyl-DL-Carnitine chloride, Parthenolide, 4-(4-Fluorophenyl)-2-(4-nitrophenyl)-5-(4-pyridyl)-1H-imidazole)(PD 169316), 1,10-Phenanthroline monohydrate, 4-Phenyl-3-furoxancarbonitrile, Prazosin hydrochloride, Protoporphyrin IX disodium, Quinacrine dihydrochloride, Quabain, Retinoic acid p-hydroxyanilide, Rottlerin, Sanguinarine chloride, Tetraethylthium disulfide and 3-[1-(3H-Imidazol-4-yl)-meth-(Z)-ylidene]-5-methoxy-1,3-dihydro-indol-2-one)(SU 9516) were added to MCF-7 SPS and CP70 sps cells for testing, and results are shown in Table 2 and Table 3. Among those compounds, Niclosamide can inhibit the formation of MCF-7 SPS. After 2 days in vitro cultivation, MCF-7 SPS cells were then treated separately with 3 μM and 30 μM Niclosamide for 48 hr, and the results indicated that Niclosamide can efficiently inhibit the proliferation and differentiation of the spheroid. The morphology and number of the spheroids were further analyzed by software image pro plus.

Additionally, MCF-7 sps and MDA-MB 231 cells were both significantly inhibited after treated with 3 μM Niclosamide (Table 2).

TABLE 2
Compounds that inhibit cancer stem cells

| Compound | Cancer cell survival (%) |
|---|---|
| 4-Phenyl-3-furoxancarbonitrile | 1.1 |
| Prazosin hydrochloride | 1.1 |
| Niclosamide | 1.4 |
| 6-Nitroso-1,2-benzopyrone | 1.5 |
| beta-Lapachone | 1.67 |
| Ouabain | 1.8 |
| Protoporphyrin IX disodium | 1.8 |
| BIO | 2.4 |
| GR 127935 hydrochloride | 2.8 |
| Quinacrine dihydrochloride | 3.8 |
| Calmidazolium chloride | 3.9 |
| Bay 11-7085 | 4 |
| Ammonium pyrrolidinedithiocarbamate | 4.4 |
| Mitoxantrone | 5.2 |
| Brefeldin A from Penicillium brefeldianum | 6.6 |
| 1,10-Phenanthroline monohydrate | 7 |
| Emetine dihydrochloride hydrate | 7.3 |
| Parthenolide | 7.3 |
| (S)-(+)-Camptothecin | 8.2 |
| CK2 Inhibitor 2 | 8.3 |
| PD 169316 | 8.8 |
| Nifedipine | 8.8 |
| Dihydroouabain | 9.1 |
| Palmitoyl-DL-Carnitine chloride | 9.7 |
| Chelerythrine chloride | 9.9 |
| CGP-74514A hydrochloride | 10 |
| Tetraethylthiuram disulfide | 10.18 |
| SU 9516 | 10.7 |
| Retinoic acid p-hydroxyanilide | 13 |
| CGS-12066A maleate | 16.3 |

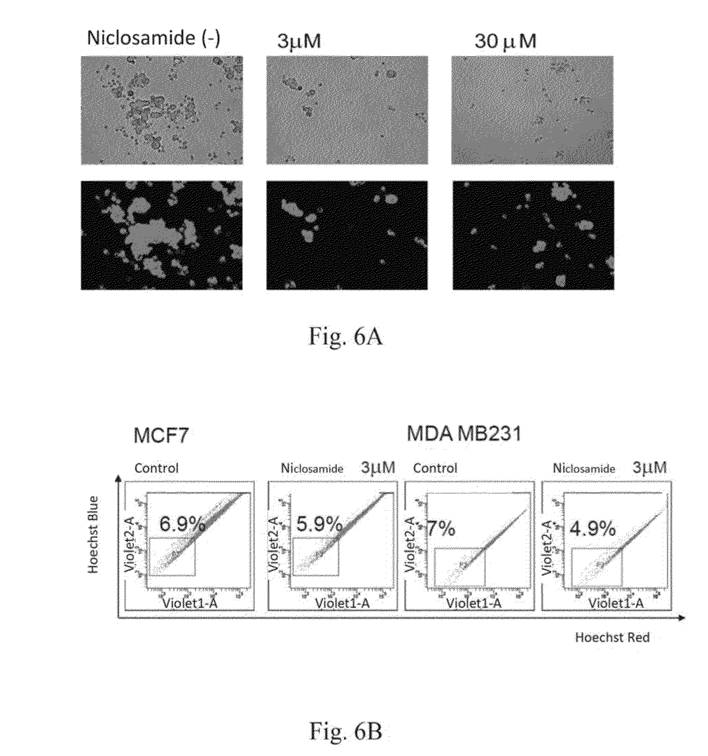

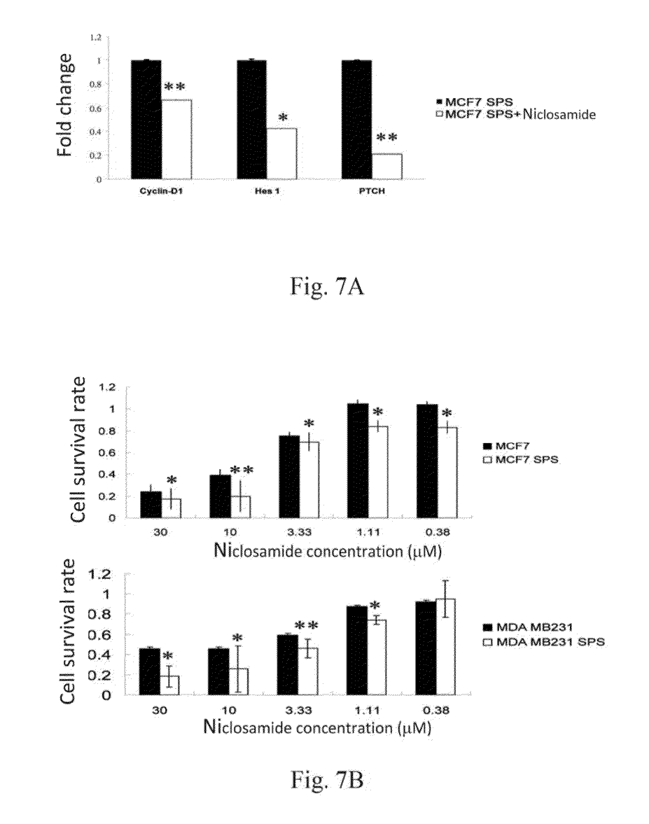

According to the results, Niclosamide significantly inhibited differentiation and proliferation of MCF-7 SPS cells, and based on the findings from proliferation dynamics experiments, FIG. 6A demonstrated that the higher the Niclosamide's concentration is or the longer the treatment time is, the better the toxicity and inhibition effects would be. In addition, from FIG. 7B, the survival of MDA-MB231 SPS cells following Niclosamide treatment is negatively correlated with the treatment.

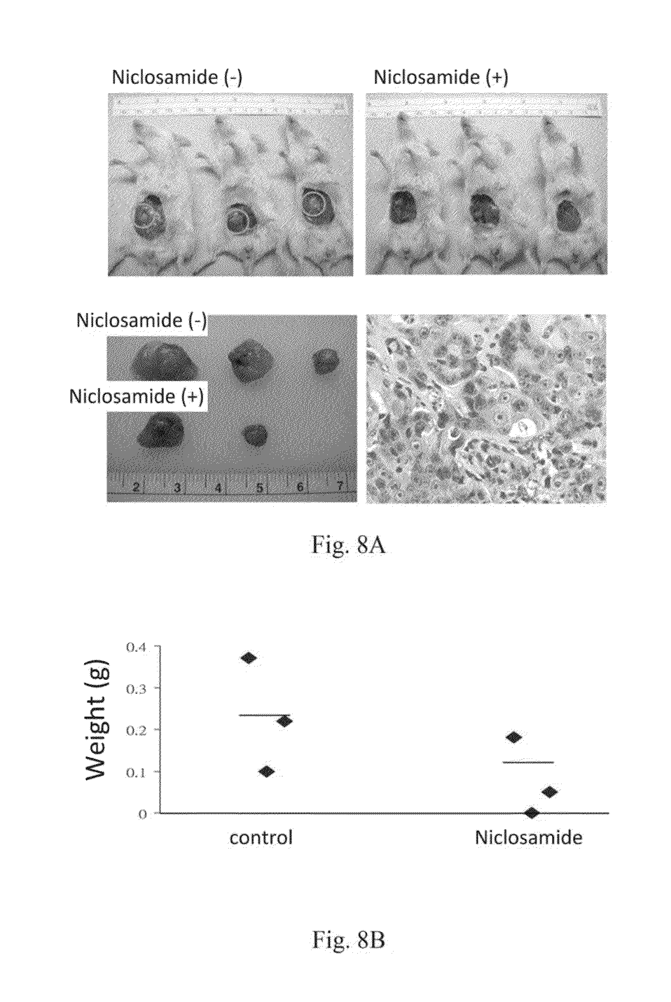

Furthermore, treating MCF-7 sps cells with Niclosamide indicated that the expression of Cyclin D1 (a protein that highly associated with breast cancer), Hes-1 (a protein that highly associated with cervical cancer), and PTCH (a protein that highly associated with pancreatic cancer) was inhibited notably by 57.4%, 33.1%, and 79.2%, respectively (as shown in FIG. 7A). On the other hand, flow cytometry analysis showed higher expression of OCT4 in MCF-7 sps cells treated with 5 μM Niclosamide (FIG. 8A). Moreover, based on the experiment showing the expression of MCF-7 sps cells in NOD/SCID mice treated with 5 μM Niclosamide, the sizes and weights of the tumors found in mice treated with Niclosamide are smaller and lighter than those found in the untreated mice (FIG. 8B).

### Example 3

### The Inhibition Effects of Niclosamide on CP70 Cells and CP70 sps Cells

Around 1200 compounds that can potentially inhibit differentiation and growth of OVCA as well as OVCA sps cells were selected from the LOPAC database and tested for their toxicity effects by MTS assay and cell survival by ATP-based assay. Table 3 shows the inhibition results of the screened compounds on OVCA sps cells.

TABLE 3
Compounds that can inhibit proliferation
and differentiation of OVCA cells

| Compound | Cancer cell survival (%) |
|---|---|
| Sanguinarine chloride | 20.37 |
| Ouabain | 26.69 |
| Diphenyleneiodonium chloride | 31.37 |
| Protoporphyrin IX disodium | 34.75 |
| Niclosamide | 34.86 |
| Rottlerin | 38.89 |
| (S)-(+)-Camptothecin | 39.11 |
| Brefeldin A from Penicillium brefeldianum | 41.18 |
| Calcimycin | 42.70 |
| (+)-Butaclamol hydrochloride | 42.81 |
| Dihydroouabain | 45.97 |
| Emetine dihydrochloride hydrate | 49.13 |
| Dequalinium dichloride | 54.68 |
| Vinblastine sulfate salt | 64.6 |

A total of 60 compounds that can potentially inhibit differentiation and growth of cancer stem cells were selected from the LOPAC database and tested for their cytotoxicity effects on OVCA as well as OVCA sps cells at the lower concentration of 3 μM. Based on the results, three compounds with the best cytotoxicity effect are identified: (+)-Butaclamol hydrochloride, Niclosamide, and Rottlerin. Among which, Niclosamide and Rottlerin are toxic to OVCA sps cells, whereas (+)-Butaclamol hydrochloride can kill both OVCA and OVCA sps cells. In pre-clinical ovarian cancer studies, Niclosamide showed successful inhibition on cancer stem cells as well.

Next, OCT-4 as well as cell surface markers such as NANOG and NESTIN were examined by flow cytometry using anti-human OCT4 and other specific antibodies. OCT4 is one of the important transcription factors expressed during the early stage of embryo development and is only expressed in pluripotent cells that are capable of differentiation. OCT4 and NANOG are key regulation factors in embryonic stem (ES) cell differentiation. On the other hand, NESTIN, an intermediate filament protein, expressed mainly in metastatic cells as well as proliferating cells, including neural stem cells, astrocytes, and immature muscle cells, during embryo development. NESTIN is commonly used for labeling differentiated cells at various periods and developmental stages. According to the results, NANOG and NESTIN expression showed significant increase in side population CP70 sps cells when compared with CP70 sp cells.

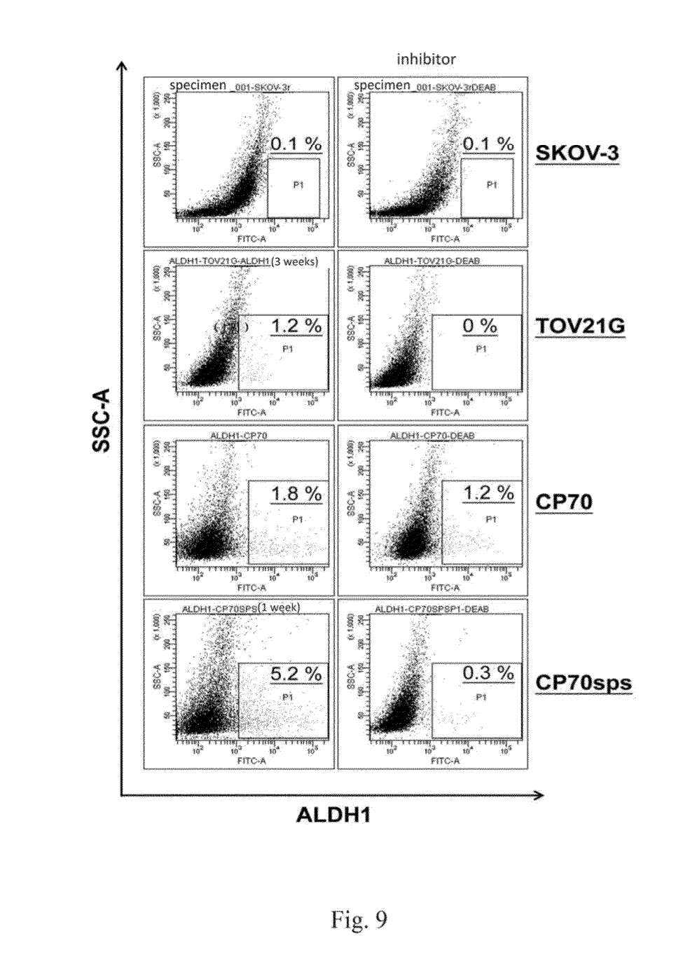

Alternatively, stem cell probes such as ABCG2 and ALDH-1 were used for identification of the transportation pores on cell membrane. The results showed that the expression of ABCG2 and ALDH-1 were notably higher in CP70 sps cells than in CP70 cells (FIG. 9). Likewise, the expression of CD34, CD44 and CD133 were all higher in CP70 sps cells than in CP70 cells as well when examined by flow cytometry using these protein markers.

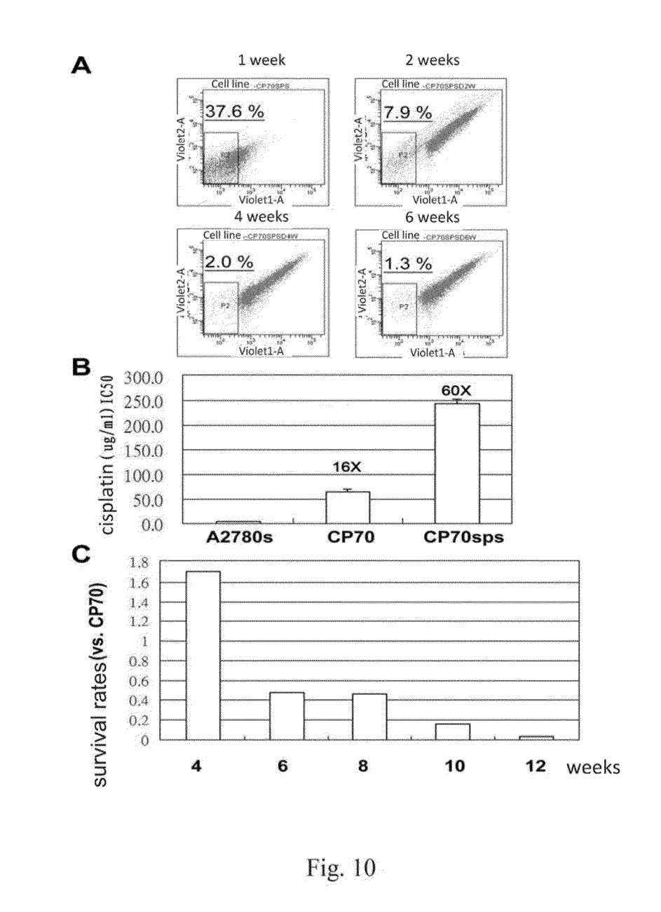

CP70 and CP70 sps cells are both differentiated from the parental cell line, A2780s. Therefore, 60 compounds that can potentially inhibit differentiation and growth of cancer stem cells were selected from the LOPAC database and tested on A2780s cells. These treated cells were compared with A2780s cells treated with the anti-cancer drug, Cisplatin, and the results are shown in FIGS. 10A, 10B, and 10C, indicating that both CP70 and CP70 sps cells were inhibited, and the survival of cancer cells decreased dramatically while the treatment time is extended.

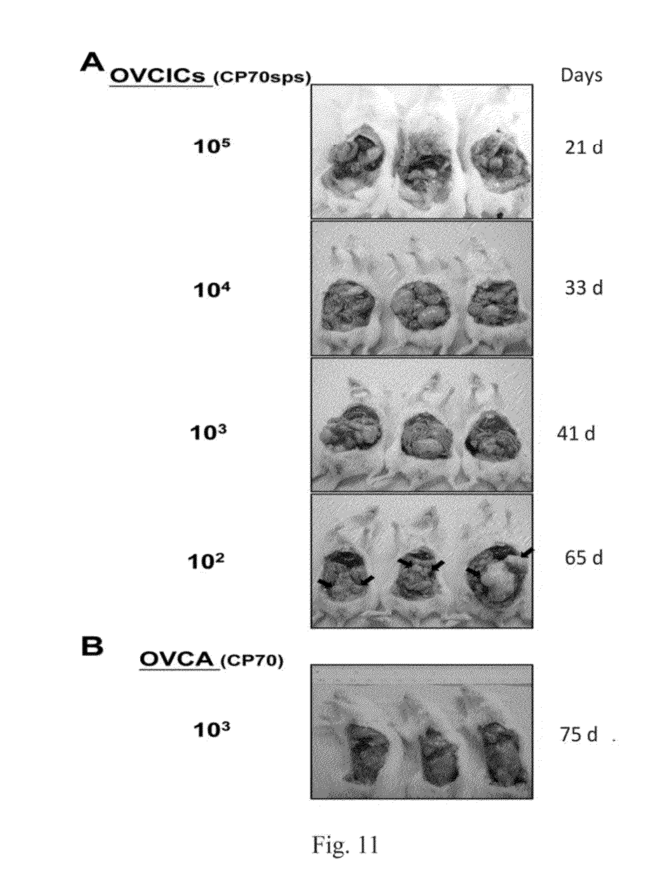

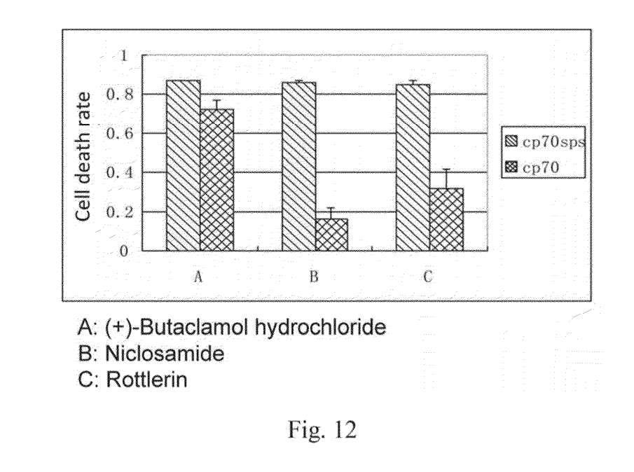

Intraperitoneal injection of NOD/SCID mice with CP70 and CP70 sps cells induced tumor formation (FIGS. 11A and 11B) in these treated mice. Results from treating CP70 and CP70 sps cells with the three most toxic compounds: Niclosamide, Rottlerin and (+)-Butaclamol hydrochloride suggested that all three compounds have significant inhibition effect on CP70 sps cells, while (+)-Butaclamol hydrochloride is also toxic to CP70 cells (FIG. 12).

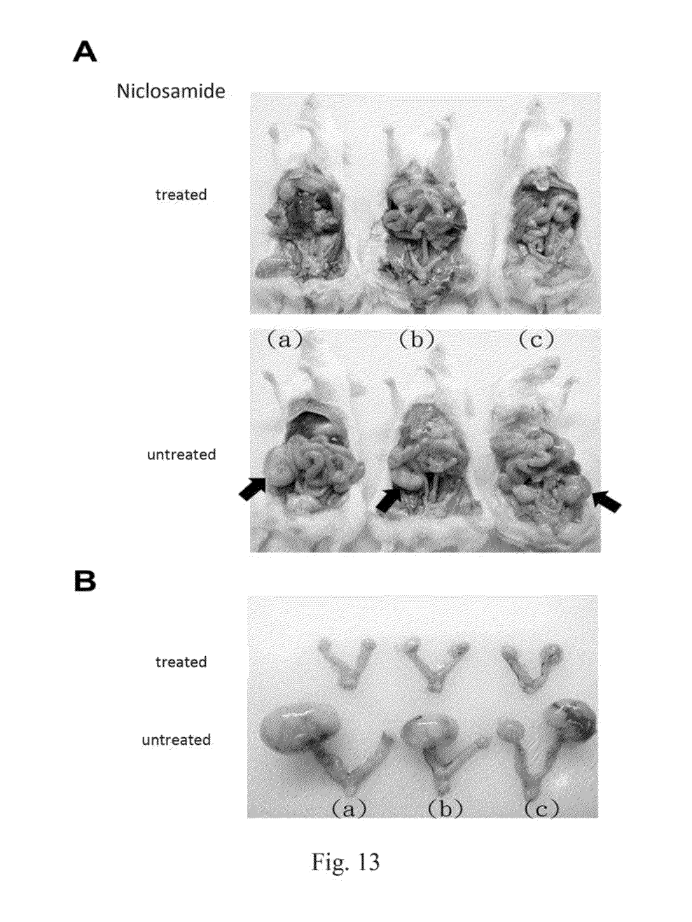

Administration of 5 μM Niclosamide to treat side population CP70 sps cells in NOD/SCID mice showed both the sizes and the weights of the tumors in the Niclosamide-treated mice are smaller and lighter than that found in the untreated mice (FIGS. 13A and 13B).

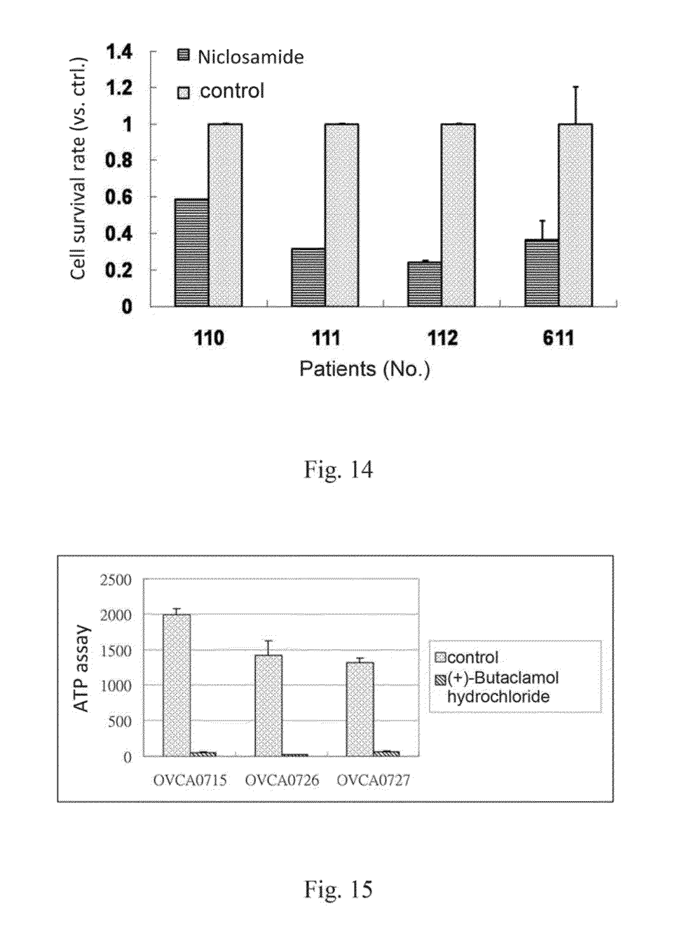

OVCICs sps cells collected from ovarian cancer patient(s) were treated with Niclosamide in vitro to test the its toxicity, and as shown in FIG. 14, Niclosamide successfully inhibited differentiation and growth of ovarian cancer cells.

OVCICs sps cells collected from ovarian cancer patient(s) were treated with (+)-Butaclamol hydrochloride in vitro to test the its toxicity, and as shown in FIG. 15, (+)-Butaclamol hydrochloride successfully inhibited differentiation and growth of ovarian cancer cells.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A pharmaceutical composition used for cancer treatment comprises (a) at least one compound, wherein the compound is selected from the following: Ammonium pyrrolidinedithiocarbamate; Bay 11-7085; (2′Z,3′E)-6-Bromoindirubin-3′-oxime)(BIO), Brefeldin A; (+)-Butaclamol; Calcimycin; Calmidazolium chloride; Chelerythrine chloride; CK2 Inhibitor 2; CGP-74514A hydrochloride; CGS-12066A meleate; Dequalinium dichloride; Dihydroouabain; Diphenyleneiodonium chloride; Emetine dihydrochloride hydrate; GR 127935 hydrochloride; beta-Lapachone; Niclosamide; Nifedipine; 6-Nitroso-1,2-benzopyrone; Palmitoyl-DL-Carnitine chloride; Parthenolide; PD 169316; 1,10-Phenanthroline monohydrate; 4-Phenyl-3-furoxancarbonitrile; Prazosin hydrochloride; Protoporphyrin IX disodium; Quinacrine dihydrochloride; Quabain; Retinoic acid p-hydroxyanilide; Rottlerin; Sanguinarine chloride; Tetraethylthium disulfide and SU9516; and (b) pharmaceutically acceptable carriers or excipients added to the pharmaceutical composition.

2. The pharmaceutical composition of claim 1, wherein the composition comprises the pharmaceutically acceptable salts or solvates of the compound, or pharmaceutically functional derivatives of the compound thereof.

3. The pharmaceutical composition of claim 1, wherein the abovementioned cancer is selected from the following cancers or their combinations thereof: cervical cancer; breast cancer; ovarian cancer; colon cancer; pancreatic cancer; stomach cancer; prostate cancer; acute T cell leukemia; leukemia; liver cancer; endometrial cancer; lung cancer; colorectal cancer; melanoma; malignant sarcoma; or other malignant tumors.

4. The pharmaceutical composition of claim 1, wherein the excipient comprises diluents; fillers; binders; disintegrants; and lubricants.

5. A pharmaceutical composition used for treating cancers comprises (a) Niclosamide and its pharmaceutically acceptable salts and solvates, or its pharmaceutically functional derivatives; and (b) pharmaceutically acceptable carriers or excipients added to the pharmaceutical composition.

6. The pharmaceutical composition of claim 5, wherein the cancer is selected from the following cancers or their combinations thereof: cervical cancer; breast cancer; ovarian cancer; colon cancer; pancreatic cancer; stomach cancer; prostate cancer; acute T cell leukemia; leukemia; liver cancer; endometrial cancer; lung cancer; colorectal cancer; melanoma; malignant sarcoma; or other malignant tumors.

7. The pharmaceutical composition of claim 5, wherein the excipient comprises diluents; fillers; binders; disintegrants; and lubricants.

8. A pharmaceutical composition used for treating cancers comprises (a) Rottlerin and its pharmaceutically acceptable salts and solvates, or its pharmaceutically functional derivatives; and (b) pharmaceutically acceptable carriers or excipients added to the pharmaceutical composition.

9. The pharmaceutical composition of claim 8, wherein the cancer is selected from the following cancers or their combinations thereof: cervical cancer; breast cancer; ovarian cancer; colon cancer; pancreatic cancer; stomach cancer; prostate cancer; acute T cell leukemia; leukemia; liver cancer; endometrial cancer; lung cancer; colorectal cancer; melanoma; malignant sarcoma; or other malignant tumors.

10. The pharmaceutical composition of claim 8, wherein the excipient comprises diluents; fillers; binders; disintegrants; and lubricants.

11. A pharmaceutical composition used for treating cancers comprises (a) (+)-Butaclamol and its pharmaceutically acceptable salts and solvates, or its pharmaceutically functional derivatives; and (b) pharmaceutically acceptable carriers or excipients added to the pharmaceutical composition.

12. The pharmaceutical composition of claim 11, wherein the cancer is selected from the following cancers or their combinations thereof: cervical cancer; breast cancer; ovarian cancer; colon cancer; pancreatic cancer; stomach cancer; prostate cancer; acute T cell leukemia; leukemia; liver cancer; endometrial cancer; lung cancer; colorectal cancer; melanoma; malignant sarcoma; or other malignant tumors.

13. The pharmaceutical composition of claim 11, wherein the excipient comprises diluents; fillers; binders; disintegrants; and lubricants.
